(19) United States
(12) Patent Application Publication
(10) Pub. No.: US 2024/0329022 A1
(43) Pub. Date: Oct. 3, 2024

(54) SENSOR ELEMENT AND GAS SENSOR

(71) Applicant: NGK INSULATORS, LTD., Nagoya-shi, Aichi (JP)

(72) Inventors: Kaoru SHIBUTANI, Nagoya-shi (JP); Ryo ONISHI, Iwakura-shi (JP)

(21) Appl. No.: 18/604,653

(22) Filed: Mar. 14, 2024

(30) Foreign Application Priority Data
Mar. 31, 2023 (JP) 2023-058160

(51) Int. Cl. G01N 33/00; G01N 27/407; G01N 27/409; G01N 27/41; G01N 27/419

(52) U.S. Cl. CPC G01N 33/0063; G01N 27/4077; G01N 27/409; G01N 27/41; G01N 27/419

(57) ABSTRACT

A sensor element includes: an element body having a front end and a back end which are both ends in a longitudinal direction, and a lateral face which is a face in the longitudinal direction; an outer lead section disposed on the lateral face; a porous layer that covers part of the lateral face; and a dense layer that covers part of the lateral face and in contact with the porous layer on the front-end side and/or the back-end side, wherein the outer lead section has a low porosity region covered by the porous layer, and a high porosity region which is covered by the dense layer, in contact with the low porosity region, and has a higher porosity than the low porosity region.

### CROSS-REFERENCE TO RELATED APPLICATION

The present application claims priority from Japanese application JP2023-058160, filed on Mar. 31, 2023, the content of which is hereby incorporated by reference into this application.

### BACKGROUND OF THE INVENTION

### 1. Field of the Invention

The present invention relates to a sensor element and a gas sensor.

### 2. Description of the Related Art

Hitherto, a sensor element that detects the concentration of a specific gas, such as NOx, in a measurement-object gas, such as the exhaust gas of an automobile, is known (see, for example, PTL 1). The sensor element of PTL 1 includes an element body, an outer electrode, a connector electrode, an outer lead section, a porous layer, and a dense layer. A front-end side of the element body is exposed to a measurement-object gas. The outer electrode is disposed on the front-end side of a lateral face of the element body. The connector electrode is disposed on a back-end side of the lateral face of the element body to be electrically connected to a contact portion of a connector. The outer lead section is disposed on the lateral face of the element body to electrically connect the outer electrode and the connector electrode. The porous layer is disposed on the lateral face of the element body, and covers and protects at least part of the outer lead section. The dense layer is disposed on the lateral face of the element body, and located on the front-end side with respect to the connector electrode so that the porous layer is divided along the longitudinal direction of the element body, or is located on the back-end side with respect to the porous layer. When the water content in the measurement-object gas moves in the porous layer toward the back-end side of the element body due to capillary action, the water is prevented from reaching the connector electrode due to the presence of the dense layer because the capillary action is unlikely to occur in the dense layer.

### CITATION LIST

### Patent Literature

PTL1: International Publication No. 2019/155865

### SUMMARY OF THE INVENTION

In a sensor element in which an outer lead section is covered by a dense layer and a porous layer as in PTL 1, crack may occur in the sensor element.

The present invention has been devised to solve such a problem, and it is a main object to prevent crack of the sensor element.

The present invention employs the following device to achieve the above-described object.

[1] The sensor element of the present invention is a sensor element for detecting a specific gas concentration in a measurement-object gas, and includes: an element body having a front end and a back end which are both ends along a longitudinal direction, and a lateral face which is a surface along the longitudinal direction, wherein a front-end side is exposed to the measurement-object gas; an outer electrode disposed on the front-end side of the lateral face; a connector electrode disposed on a back-end side of the lateral face to be electrically connected to an outside; an outer lead section disposed on the lateral face to electrically connect the outer electrode and the connector electrode; a porous layer that covers part of the lateral face; and a dense layer that covers part of the lateral face, the dense layer being disposed on the back-end side with respect to at least part of the porous layer, disposed on the front-end side with respect to the connector electrode, and in contact with the porous layer on the front-end side and/or the back-end side. The outer lead section has a low porosity region covered by the porous layer, and a high porosity region which is covered by the dense layer, in contact with the low porosity region, and has a higher porosity than the low porosity region.

In the sensor element, the outer lead section has a low porosity region covered by the porous layer, and a high porosity region which is covered by the dense layer, in contact with the low porosity region, and has a higher porosity than the low porosity region. Because of this, the occurrence of crack of the sensor element can be prevented. The inventors confirmed this by experiments and analysis.

[2] In the sensor element described above (the sensor element according to [1] stated above), the high porosity region may have a porosity less than or equal to 40%. In this configuration, movement of water inside the high porosity region can prevent the water from coming around the dense layer and moving to the back-end side of the element body. Thus, water can be prevented from reaching the connector electrode.

[3] In the sensor element described above (the sensor element according to [1] or [2] stated above), the high porosity region may have a porosity less than 15%. In this configuration, movement of water inside the high porosity region can further prevent the water from coming around the dense layer and moving to the back-end side of the element body.

[4] In the sensor element described above (the sensor element according to any one of [1] to [3] stated above), the difference in porosity between the high porosity region and the low porosity region may be greater than or equal to 18. In this configuration, the effect of preventing the occurrence of crack of the sensor element is more reliably obtained by providing the above-mentioned high porosity region and low porosity region.

[5] In the sensor element described above (the sensor element according to any one of [1] to [4] stated above), the high porosity region may have a porosity greater than or equal to 5%. In this configuration, the effect of preventing the occurrence of crack of the sensor element is more reliably obtained by providing the above-mentioned high porosity region and low porosity region.

[6] In the sensor element described above (the sensor element according to any one of [1] to [5] stated above), the dense layer may have a thickness less than or equal to 10 μm. In this configuration, the occurrence of crack of the sensor element can be further prevented.

[7] In the sensor element described above (the sensor element according to any one of [1] to [6] stated above), the dense layer may have an overlapping section at a portion in contact with the porous layer, the overlapping section overlapping with the porous layer in a thickness direction of the dense layer, and the dense layer may cover only the high porosity region of the high porosity region and the low porosity region over a length from the front-end side to the back-end side of the dense layer including the overlapping section.

[8] In the sensor element described above (the sensor element according to any one of [1] to [7] stated above), the high porosity region may have a protruding portion which protrudes from the dense layer to the front-end side and/or the back-end side, the protruding portion being covered by the porous layer, and the protruding portion may have a length less than or equal to 0.5 mm along the longitudinal direction. In this configuration, the effect of preventing the occurrence of crack of the sensor element is more reliably obtained by providing the above-mentioned high porosity region and low porosity region.

[9] In the sensor element described above (the sensor element according to any one of [1] to [8] stated above), the outer lead section may contain zirconia. Here, when the outer lead section contains zirconia, the zirconia expands due to TM transformation, and the outer lead section is deformed, thus crack of the sensor element tends to occur. Therefore, when the outer lead section contains zirconia, it is significant to prevent the occurrence of crack of the sensor element by providing the outer lead section with the low porosity region and the high porosity region.

[10] The gas sensor of the present invention includes the sensor element according to any one of [1] to [9] stated above. Thus, with the gas sensor, the same effect as that of the above-described sensor element, for example, the effect of preventing the occurrence of crack of the sensor element is obtained.

### BRIEF DESCRIPTION OF THE DRAWINGS

FIG. 1 is a vertical cross-sectional view illustrating the manner in which a gas sensor 10 is attached to a pipe 58.

FIG. 2 is a perspective view of a sensor element 20.

FIG. 3 is a vertical cross-sectional view of the sensor element 20.

FIG. 4 is a top view of the sensor element 20.

FIG. 5 is a vertical cross-sectional view illustrating the periphery of a high porosity region 77 covered by a first dense layer 92.

FIG. 6 is a cross-sectional view illustrating the periphery of a first dense layer 92 and a high porosity region 77 in a modification.

FIG. 7 is a cross-sectional view illustrating the periphery of a first dense layer 92 and a high porosity region 77 in a modification.

FIG. 8 is a cross-sectional view illustrating the periphery of a first dense layer 92 and a high porosity region 77 in a modification.

FIG. 9 is a cross-sectional view illustrating the periphery of a first dense layer 92 and a high porosity region 77 in a modification.

### DETAILED DESCRIPTION OF THE INVENTION

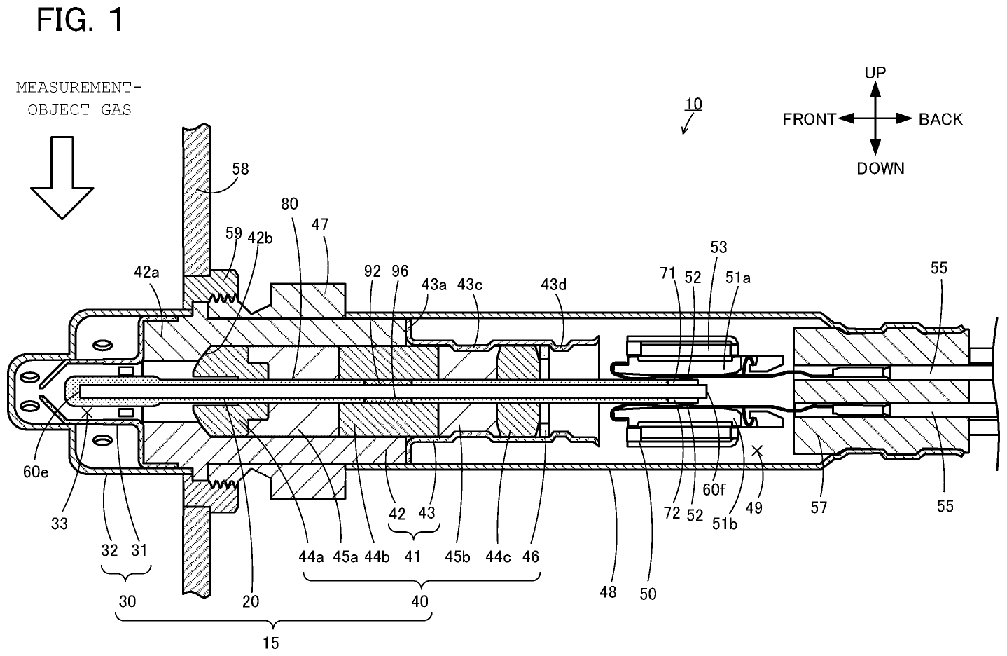

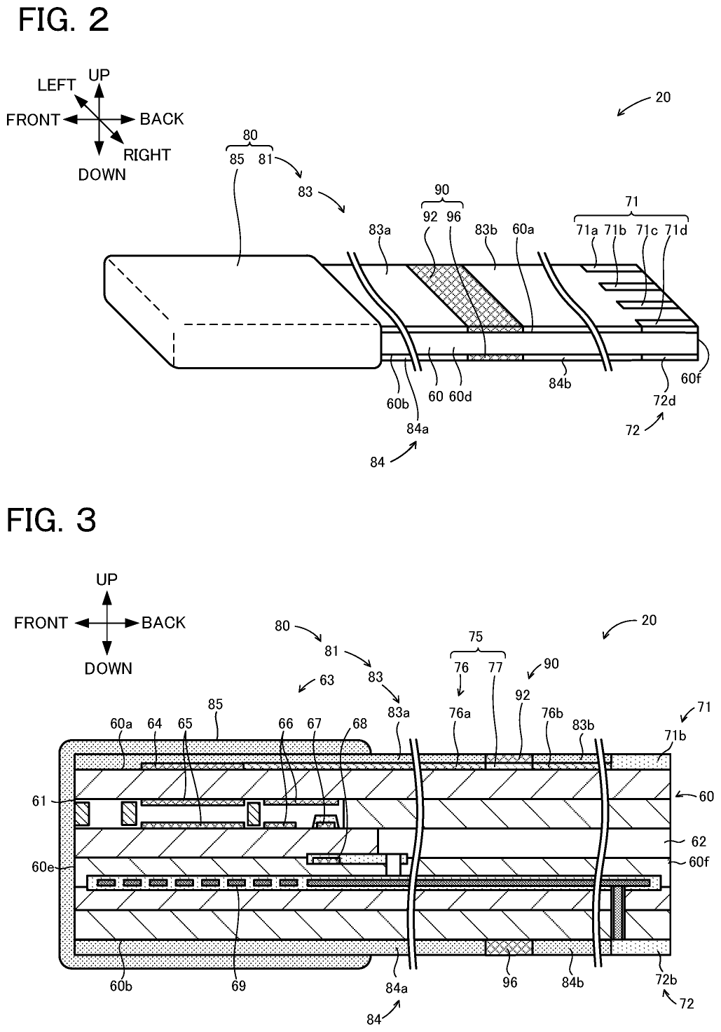

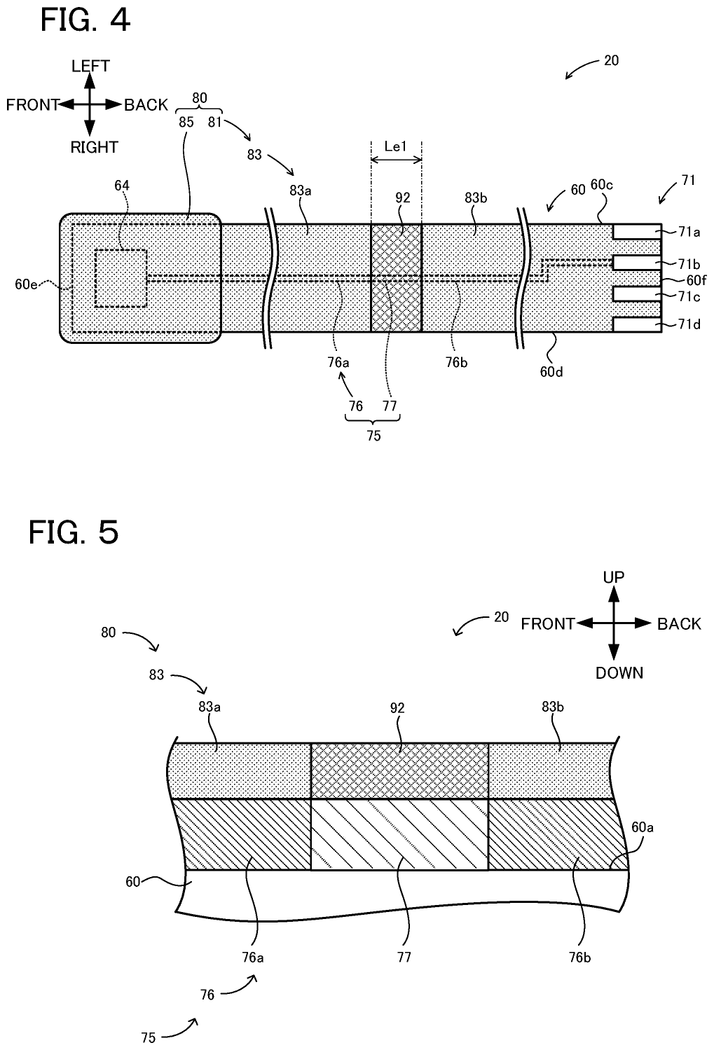

Next, an embodiment of the present invention will be described using the drawings. FIG. 1 is a vertical cross-sectional view illustrating the manner in which a gas sensor 10 as an embodiment of the present invention is attached to a pipe 58. FIG. 2 is a perspective view of a sensor element 20 seen from the front upper right. FIG. 3 is a vertical cross-sectional view schematically illustrating a vertical cross-section of the sensor element 20. FIG. 4 is a top view of the sensor element 20. FIG. 5 is a vertical cross-sectional view illustrating the periphery of a high porosity region 77 of an outer lead 75 covered by a first dense layer 92. In the present embodiment, as illustrated in FIG. 1 to FIG. 5, it is assumed that the longitudinal direction of an element body 60 of the sensor element 20 is the front-back direction (length direction), the stacking direction (thickness direction) of the solid electrolyte layer of the element body 60 is the up-down direction, and the direction perpendicular to the front-back direction and the up-down direction is the left-right direction (width direction).

As shown in FIG. 1, the gas sensor 10 includes an assembly 15, a nut 47, an external cylinder 48, a connector 50, lead wires 55, and a rubber stopper 57. The assembly 15 includes the sensor element 20, a protective cover 30, and an element-sealing member 40. The gas sensor 10 is attached to the pipe 58 such as an exhaust gas pipe of a vehicle and used to measure the concentration of a specific gas (a specific gas concentration) such as NOx or O₂ contained in the exhaust gas used as a measurement-object gas. In the present embodiment, the gas sensor 10 measures the concentration of NOx as the specific gas concentration. The sensor element 20 has opposite ends (front and back ends) along the longitudinal direction, and the front-end side is the side exposed to the measurement-object gas.

As illustrated in FIG. 1, the protective cover 30 includes a bottomed cylindrical inner protective cover 31 that covers the front-end side of the sensor element 20, and a bottomed cylindrical outer protective cover 32 that covers the inner protective cover 31. A plurality of holes to allow a measurement-object gas to flow therethrough are formed in each of the inner protective cover 31 and the outer protective cover 32. An element chamber 33 is formed as the space surrounded by the inner protective cover 31, and a fifth face 60e (front-end face) of the sensor element 20 is disposed in the element chamber 33.

The element-sealing member 40 is a member for sealing and fixing the sensor element 20. The element-sealing member 40 includes: a cylindrical body 41 including a metal fitting 42 and an inner cylinder 43; insulators 44a to 44c (examples of the dense body); compacts 45a and 45b; and a metal ring 46. The sensor element 20 is disposed so as to extend along the center axis of the element-sealing member 40 (an axis extending in the front-back direction in the present embodiment) and pierces through the element-sealing member 40 in the axial direction.

The metal fitting 42 is a cylindrical metallic member. The metal fitting 42 has a thick-walled portion 42a located on the front side and having an inner diameter smaller than that of the back side. The protective cover 30 is attached to a portion of the metal fitting 42 that is on the same side as the front end of the sensor element 20 (i.e., the front side). The back end of the metal fitting 42 is welded to a flange portion 43a of the inner cylinder 43. A part of the inner circumferential surface of the thick-walled portion 42a is formed as a bottom surface 42b that is a step surface. The bottom surface 42b bears the insulator 44a such that the insulator 44a does not protrude forward. The metal fitting 42 has a through hole that passes through the metal fitting 42 in the axial direction (the front-back direction in the present embodiment), and the sensor element 20 passes through the through hole.

The inner cylinder 43 is a cylindrical metallic member and has the flange portion 43a at its front end. The inner cylinder 43 and the metal fitting 42 are welded to each other so as to be coaxial with each other. The inner cylinder 43 has a reduced diameter portion 43c for pressing the compact 45b in a direction toward the center axis of the inner cylinder 43 and a reduced diameter portion 43d for pressing the insulators 44a to 44c and the compacts 45a and 45b in the front direction through the metal ring 46. The inner cylinder 43 has a through hole that passes through the inner cylinder 43 in the axial direction (the front-back direction in the present embodiment), and the sensor element 20 passes through the through hole. The through hole of the metal fitting 42 and the through hole of the inner cylinder 43 are in communication with each other in the axial direction and form the through hole of the cylindrical body 41.

The insulators 44a to 44c and the compacts 45a and 45b are disposed between the inner circumferential surface of the through hole of the cylindrical body 41 and the sensor element 20. The insulators 44a to 44c serve as supporters for the compacts 45a and 45b. Examples of the material of the insulators 44a to 44c include ceramics such as alumina, steatite, zirconia, spinel, cordierite, and mullite and glass. The insulators 44a to 44c are dense members, and their porosity is, for example, less than 1%. Each of the insulators 44a to 44c is a hollow columnar member having a through hole that passes therethrough in the axial direction (the front-back direction in the present embodiment), and the sensor element 20 passes through the through hole. In the present embodiment, the through hole of each of the insulators 44a to 44c has a quadrilateral cross-section that is perpendicular to the axial direction and conforms to the shape of the sensor element 20. The compacts 45a and 45b are formed, for example, by molding a powder and each serve as a sealing medium. Examples of the material of the compacts 45a and 45b include talc and ceramic powders such as alumina powder and boron nitride powder, and the compacts 45a and 45b may each contain at least one of these materials. Particles included in the compacts 45a and 45b may have an average particle diameter of 150 to 300 μm. The compact 45a is filled between the insulators 44a and 44b, sandwiched therebetween from opposite sides (front and back sides) in the axial direction, and pressed by the insulators 44a and 44b. The compact 45b is filled between the insulators 44b and 44c, sandwiched therebetween from opposite sides (front and back sides) in the axial direction, and pressed by the insulators 44b and 44c. The insulators 44a to 44c and the compacts 45a and 45b are sandwiched between the bottom surface 42b of the thick-walled portion 42a of the metal fitting 42 and both the reduced diameter portion 43d and the metal ring 46 and pressed in the axial direction from opposite sides (the front and back sides). The pressing force applied by the reduced diameter portions 43c and 43d causes the compacts 45a and 45b to be compressed between the cylindrical body 41 and the sensor element 20, and the compacts 45a and 45b close the communication between the element chamber 33 in the protective cover 30 and a space 49 in the external cylinder 48 and fix the sensor element 20.

The nut 47 is fixed to the outer side of the metal fitting 42 so as to be coaxial with the metal fitting 42. The nut 47 has a male thread portion formed on the outer circumferential surface of the nut 47. The male thread portion is screwed into a female thread portion formed on the inner circumferential surface of a fixing member 59 welded to the pipe 58. In this manner, the gas sensor 10 is fixed to the pipe 58 with the front-end side of the sensor element 20 and the protective cover 30 protruding into the pipe 58.

The external cylinder 48 is a cylindrical body made of metal, and covers the inner cylinder 43, the back-end side of the sensor element 20, and the connector 50. The back end of the main metal fitting 42 is inserted inside the external cylinder 48. The front end of the external cylinder 48 is welded to the main metal fitting 42. A plurality of leads 55 connected to the connector 50 are drawn to the outside from the back end of the external cylinder 48. The connector 50 has housings 51a, 51b, a plurality of contact metal fittings 52, and clamps 53. The housings 51a, 51b are members made of ceramics, such as alumina, and disposed at upper and lower positions of the back end of the sensor element 20. The multiple contact metal fittings 52 are each a member made of metal, and disposed on the lower side of the housing 51a and on the upper side of the housing 51b. The clamps 53 are members obtained by bending plate-shaped metal into C-character shape, and vertically hold the housings 51a, 51b with an elastic force to press them in a direction in which they approach each other. The elastic force from the clamps 53 causes the housings 51a, 51b to hold and fix the back end of the sensor element 20 via a plurality of contact metal fittings 52. The plurality of contact metal fittings 52 are each in contact with and electrically connected to corresponding one of a plurality of upper connector electrodes 71 and a plurality of lower connector electrodes 72 disposed on the surface of the back-end side of the sensor element 20, and electrically connected to corresponding one of a plurality of leads 55. The plurality of leads 55 are each electrically connected to one of a plurality of electrodes 64 to 68 inside the sensor element 20 and a heater 69 via one of the connector 50, and the plurality of upper connector electrodes 71 and the plurality of lower connector electrodes 72. The gap between the external cylinder 48 and the leads 55 are sealed by the rubber stopper 57. The space 49 in the external cylinder 48 is filled with a reference gas. A sixth face 60f (back-end face) of the sensor element 20 is disposed in the space 49.

As illustrated in FIG. 2 to FIG. 4, the sensor element 20 includes the element body 60, a detector 63, the heater 69, the plurality of upper connector electrodes 71, the plurality of lower connector electrodes 72, a porous layer 80, and a dense layer 90. The element body 60 has a layered body obtained by stacking multiple (six in FIG. 3) oxygen-ion-conductive solid electrolyte layers such as zirconia (ZrO₂) layers. The element body 60 has a rectangular parallelepiped shape with the longitudinal direction in the front-back direction, and has first to sixth faces 60a to 60f as the outer surfaces on the up, down, left, right, front, and back. The first to fourth faces 60a to 60d are the surfaces along the longitudinal direction of the element body 60, and correspond to the lateral faces of the element body 60. The fifth face 60e is the front-end face of the element body 60, and the sixth face 60f is the back-end face of the element body 60. The dimensions of the element body 60 may be, for example, such that the length in the front-back direction is 25 mm or more and 100 mm or less, the width in the left-right direction is 2 mm or more and 10 mm or less, and the thickness in the up-down direction is 0.5 mm or more and 5 mm or less. In the element body 60, a measurement-object gas inlet 61 and a reference gas inlet 62 are formed, the measurement-object gas inlet 61 being open in the fifth face 60e to allow a measurement-object gas to be introduced to the inside of the element body 60, the reference gas inlet 62 being open in the sixth face 60f to allow a reference gas (here, the atmosphere) which serves as a reference for detection of a specific gas concentration to be introduced to the inside of the element body 60.

The detector 63 is used to detect the specific gas concentration in the measurement-object gas. The detector 63 includes a plurality of electrodes disposed on a front-end side of the element body 60. In the present embodiment, the detector 63 includes an outer electrode 64 disposed on the first face 60a and further includes an inner main pump electrode 65, an inner auxiliary pump electrode 66, a measurement electrode 67, and a reference electrode 68 that are disposed inside the element body 60. The inner main pump electrode 65 and the inner auxiliary pump electrode 66 are disposed on the inner circumferential surface of an internal space of the element body 60 and each have a tunnel-like structure.

The principle of the detection of the specific gas concentration in the measurement-object gas by the detector 63 is well known, and its detailed description will be omitted. The detector 63 detects the specific gas concentration, for example, in the following manner. The detector 63 pumps oxygen in the measurement-object gas around the inner main pump electrode 65 to the outside (the element chamber 33) or pumps oxygen from the outside according to a voltage applied between the outer electrode 64 and the inner main pump electrode 65. Moreover, the detector 63 pumps oxygen in the measurement-object gas around the inner auxiliary pump electrode 66 to the outside (the element chamber 33) or pumps oxygen from the outside according to a voltage applied between the outer electrode 64 and the inner auxiliary pump electrode 66. This allows the measurement-object gas whose oxygen concentration has been adjusted to a prescribed concentration to reach the measurement electrode 67. The measurement electrode 67 functions as a NOx reduction catalyst and reduces the specific gas (NOx) in the measurement-object gas that has reached the measurement electrode 67. Then the detector 63 generates an electric signal corresponding to an electromotive force generated between the measurement electrode 67 and the reference electrode 68 according to the oxygen concentration in the reduced gas or corresponding to a current flowing between the measurement electrode 67 and the outer electrode 64 according to the electromotive force. The electric signal generated by the detector 63 is a signal indicating a value corresponding to the specific gas concentration in the measurement-object gas (a value from which the specific gas concentration can be derived) and corresponds to the detection value detected by the detector 63.

The heater 69 is an electric resistor disposed inside the element body 60. When electric power is supplied to the heater 69 from the outside, the heater 69 generates heat and heats the element body 60. The heater 69 can heat the solid electrolyte layers included in the element body 60, can keep them hot, and can adjust their temperature to the temperature at which the solid electrolyte layers are activated (e.g., 800° C.).

The plurality of upper connector electrodes 71 and the plurality of lower connector electrodes 72 are each disposed on the back-end side of one of the lateral faces of the element body 60, and serves as an electrode to be electrically connected to the outside. The plurality of upper connector electrodes 71 and the plurality of lower connector electrodes 72 are exposed without being covered by the porous layer 80. In the present embodiment, four upper connector electrodes 71 (71a to 71d in FIG. 4) are arranged in the left-right direction, and disposed on the back-end side of the first face 60a (the upper face). Four lower connector electrodes 72 are arranged in the left-right direction, and disposed on the back-end side of the second face 60b (the lower face) opposed to the first face 60a (the upper face). The four upper connector electrodes 71 (71a to 71d) and the four lower connector electrodes 72 are each electrically connected to one of the plurality of electrodes 64 to 68 of the detector 63 and the heater 69. In the present embodiment, the upper connector electrode 71a is conductive to the measurement electrode 67, the upper connector electrode 71b is conductive to the outer electrode 64, the upper connector electrode 71c is conductive to the inner auxiliary pump electrode 66, the upper connector electrode 71d is conductive to the inner main pump electrode 65, three lower connector electrodes 72 are each conductive to the heater 69, and one lower connector electrode 72 is conductive to the reference electrode 68. The upper connector electrode 71b and the outer electrode 64 are conductive via the outer lead 75 disposed in the first face 60a (see FIG. 3 and FIG. 4). Other connector electrodes are each conductive to a corresponding electrode or the heater 69 via a lead or a through-hole disposed inside the element body 60.

The outer lead 75 is a conductor containing, for example, noble metal such as platinum (Pt) or high melting point metal such as tungsten (W), molybdenum (Mo). The outer lead 75 is preferably a cermet conductor containing noble metal or high melting point metal, and the oxygen-ion-conductive solid electrolyte (zirconia in the present embodiment) contained in the element body 60. In the present embodiment, the outer lead 75 is assumed to be a cermet conductor containing platinum and zirconia. As illustrated in FIG. 3, FIG. 4, and FIG. 5, the outer lead 75 has a low porosity region 76 and a high porosity region 77. In the present embodiment, the outer lead 75 is configured such that the portion other than the high porosity region 77 is all the low porosity region 76. The low porosity region 76 is covered by the porous layer 80, more specifically, as illustrated in FIG. 3, is covered by a first inner porous layer 83. The low porosity region 76 has a front-end side portion 76a disposed front of the high porosity region 77 to be in contact with the high porosity region 77 in the front-back direction, and a back-end side portion 76b disposed backward of the high porosity region 77 to be in contact with the high porosity region 77 in the front-back direction. The front-end side portion 76a of the low porosity region 76 is covered by a front-end side portion 83a of the first inner porous layer 83 of the porous layer 80, and a back-end side portion 76b is covered by a back-end side portion 83b of the first inner porous layer 83. The high porosity region 77 is covered by the dense layer 90, more specifically, as illustrated in FIG. 3, is covered by the first dense layer 92. As illustrated in FIG. 3 and FIG. 5, the first dense layer 92 and the high porosity region 77 have the same length in the front-back direction, and the font-end positions and the back-end positions are the same. Thus, the high porosity region 77 does not have a protruding portion which protrudes from the first dense layer 92 to the front-end side and/or the back-end side. The high porosity region 77 has a higher porosity than the low porosity region 76. The porosity of the high porosity region 77 may be greater than or equal to 5%. The porosity of the high porosity region 77 may be less than or equal to 40%, or less than 15%. It is sufficient that the porosity of the low porosity region 76 be lower than the porosity of the high porosity region 77, and the difference in porosity between the high porosity region 77 and the low porosity region 76 may be greater than or equal to 1%. The front-end side portion 76a and the back-end side portion 76b of the low porosity region 76 may have different porosities. The low porosity region 76 and the high porosity region 77 may be made of different materials from each other; however, in the present embodiment, the low porosity region 76 and the high porosity region 77 are configured to have the same material and different porosities. The line width (width) of the outer lead 75 may be, e.g., 0.1 mm or greater and 1.0 mm or less. The line thickness (thickness) of the outer lead 75 may be e.g., 1 μm or greater and 30 μm or less. An insulating layer which is not illustrated, for insulating the outer lead 75 from the solid electrolyte layer of the element body 60 may be disposed between the outer lead 75 and the first face 60a of the element body 60.

The porosities of the low porosity region 76 and the high porosity region 77 of the outer lead 75 are the values derived as follows using an image (SEM image) obtained through observation using a scanning electron microscope (SEM). First, the sensor element 20 is cut in the thickness direction of the outer lead 75 so that the cross section of the outer lead 75 serves as an observation surface, and the cut surface is embedded in a resin and polished to produce a sample for observation. Subsequently, the magnification of the SEM is set in a range from 1000 times to 10000 times, and a SEM image of the outer lead 75 is obtained by capturing the observation surface of the sample for observation. Next, image analysis is performed on the obtained image, and from the brightness distribution of brightness data of the pixels in the image, a threshold is determined by a discriminant analysis method (Otsu's binarization). Subsequently, each pixel in the image is binarized to an object portion and a pore portion based on the determined threshold to calculate the area of the object portion and the area of the pore portion. The ratio of the area of the pore portion to the entire area (the total area of the object portion and the pore portion) is derived as the porosity (unit is %). The porosity of the low porosity region 76 is set as the value which is measured in a region of the low porosity region 76, the region being in the vicinity of the high porosity region 77, for example, at a cross section which serves as the observation surface at a position with a distance of 1 mm from the high porosity region 77 in the front-back direction. The porosity of the high porosity region 77 is set as the value which is measured in a region of the high porosity region 77, the region being in the vicinity of the low porosity region 76, for example, at a cross section which serves as the observation surface at a position with a distance of 1 mm from the low porosity region 76 in the front-back direction. The porosities of the later-described porous layer 80 and dense layer 90 are also the values derived in the same manner.

The porous layer 80 is a porous body that covers part of the lateral faces of the element body 60, on which the upper, lower connector electrodes 71, 72 are disposed, that is, part of the first, second faces 60a, 60b. In the present embodiment, the porous layer 80 covers at least the front-end side of the first, second faces 60a, 60b. The porous layer 80 includes an inner porous layer 81, and an outer porous layer 85 that cover the first, second faces 60a, 60b, respectively, the outer porous layer 85 being disposed outside the inner porous layer 81.

The inner porous layer 81 includes the first inner porous layer 83 that covers the first face 60a, and a second inner porous layer 84 that covers the second face 60b. The first inner porous layer 83 has the front-end side portion 83a and the back-end side portion 83b (see FIG. 2 to FIG. 4, FIG. 5). The front-end side portion 83a covers the region from the front end of the first face 60a to the front end of the first dense layer 92 on the first face 60a. The back-end side portion 83b covers the region from the back end of the first dense layer 92 to the back end of the first face 60a on the first face 60a, the region excluding the area where the upper connector electrodes 71 are present. The right and left width of the front-end side portion 83a and the back-end side portion 83b of the first inner porous layer 83 is the same as the right and left width of the first face 60a, and the front-end side portion 83a and the back-end side portion 83b cover the first face 60a over a length from the left end to the right end of the first face 60a. The first inner porous layer 83 covers at least part of each of the outer electrode 64 and the outer lead 75. In the present embodiment, as illustrated in FIG. 3 and FIG. 4, the first inner porous layer 83 covers all the outer electrode 64, and covers the portion (in other words, all the low porosity region 76) of the outer lead 75, excluding the high porosity region 77. The first inner porous layer 83 protects the outer electrode 64 and the outer lead 75 against e.g., the components, such as sulfuric acid, in the measurement-object gas to play a role as a protective layer to prevent corrosion of these.

The second inner porous layer 84 has a front-end side portion 84a and a back-end side portion 84b (see FIG. 2, FIG. 3). The front-end side portion 84a covers the region from the front end of the second face 60b to the front end of the second dense layer 96 on the second face 60b. The back-end side portion 84b covers the region from the back end of the second dense layer 96 to the back end of the second face 60b on the second face 60b, the region excluding the area where the lower connector electrodes 72 are present. The right and left width of the front-end side portion 84a and the back-end side portion 84b of the second inner porous layer 84 is the same as the right and left width of the second face 60b, and the front-end side portion 84a and the back-end side portion 84b cover the second face 60b over a length from the left end to the right end of the second face 60b.

The outer porous layer 85 covers at least part of each of the first to fifth faces 60a to 60e. For the first face 60a and the second face 60b, the outer porous layer 85 covers the inner porous layer 81, thereby covering these faces. The outer porous layer 85 has a shorter length in the front-back direction than the inner porous layer 81, and unlike the inner porous layer 81, covers only the front end of the element body 60 and the region in the vicinity of the front end. Thus, the outer porous layer 85 covers the peripheral portion of a plurality of electrodes 64 to 68 of the detector 63 of the element body 60, in other words, a portion disposed in the element chamber 33 of the element body 60, the portion to be exposed to the measurement-object gas. Thus, the outer porous layer 85 plays a role as a protective layer to prevent the occurrence of crack of the element body 60 due to, for example, adherence of water in the measurement-object gas to the element body 60.

The porous layer 80 is formed of a ceramic porous body such as an alumina porous body, a zirconia porous body, a spinel porous body, a cordierite porous body, a titania porous body, and a magnesia porous body. In the present embodiment, the porous layer 80 is assumed to be formed of an alumina porous body. The thickness of each of the first inner porous layer 83 and the second inner porous layer 84 may be e.g., 5 μm or greater and 40 μm or less. The thickness of the outer porous layer 85 may be e.g., 40 μm or greater and 800 μm of less. The porosity of the porous layer 80 is greater than or equal to 10%. Although the porous layer 80 covers the outer electrode 64 and the measurement-object gas inlet 61, if the porosity of the porous layer 80 is greater than or equal to 10%, the measurement-object gas can pass through the porous layer 80. The porosity of the inner porous layer 81 may be 10% or greater and 50% or less. The porosity of the outer porous layer 85 may be 10% or greater and 85% or less. The outer porous layer 85 may have the same porosity as that of the inner porous layer 81, or may have a higher porosity than the inner porous layer 81.

The dense layer 90 inhibits the capillary action of water along the longitudinal direction of the element body 60. In the present embodiment, the dense layer 90 has the first dense layer 92 and the second dense layer 96. The first dense layer 92 is provided in the first face 60a in which the upper connector electrodes 71 and the first inner porous layer 83 are disposed. The first dense layer 92 is provided on the back-end side of the element body 60 with respect to at least part (here, the outer porous layer 85 and the front-end side portion 83a) of the porous layer 80, in other words, backward of the element body 60. The first dense layer 92 is provided on the front-end side of the element body 60 with respect to the plurality of upper connector electrodes 71, in other words, front of the plurality of upper connector electrodes 71. The first dense layer 92 is provided backward of the outer electrode 64. The first dense layer 92 is provided backward of any of the plurality of electrodes 64 to 68 of the detector 63 including the outer electrode 64 (see FIG. 3). When water moves backward within the first inner porous layer 83 due to capillary action, the first dense layer 92 prevents the water from passing therethrough, thereby playing a role of preventing the water from reaching the upper connector electrodes 71. The first dense layer 92 is a dense layer having a porosity less than 10%. The right and left width of the first dense layer 92 is the same as the right and left width of the first face 60a, and the first dense layer 92 covers the first face 60a over a length from the left end to the right end of the first face 60a. The front end of the first dense layer 92 is in contact with the back end of the front-end side portion 83a of the first inner porous layer 83. The back end of the first dense layer 92 is in contact with the front end of the back-end side portion 83b of the first inner porous layer 83. As illustrated in FIG. 3, FIG. 4 and FIG. 5, the first dense layer 92 covers the high porosity region 77 of the outer lead 75.

The second dense layer 96 is provided in the second face 60b in which the plurality of lower connector electrodes 72 and the second inner porous layer 84 are disposed. The second dense layer 96 is provided on the back-end side of the element body 60, in other words, backward of the element body 60 with respect to at least part (here, the outer porous layer 85 and the front-end side portion 84a) of the porous layer 80. The second dense layer 96 is provided on the front-end side of the element body 60, in other words, front of the plurality of lower connector electrodes 72 with respect to the plurality of lower connector electrodes 72. The second dense layer 96 is provided backward of any of the plurality of electrodes 64 to 68 of the detector 63 including the outer electrode 64 (see FIG. 3). When water moves backward within the second inner porous layer 84 due to capillary action, the second dense layer 96 prevents the water from passing therethrough, thereby playing a role of preventing the water from reaching the lower connector electrodes 72. The second dense layer 96 is a dense layer having a porosity less than 10%. The right and left width of the second dense layer 96 is the same as the right and left width of the second face 60b, and the second dense layer 96 covers the second face 60b over a length from the left end to the right end of the second face 60b. The front end of the second dense layer 96 is in contact with the back end of the front-end side portion 84a of the second inner porous layer 84. The back end of the second dense layer 96 is in contact with the front end of the back-end side portion 84b of the second inner porous layer 84.

The first dense layer 92 and the second dense layer 96 differ from the porous layer 80 in that the porosity is less than 10%; however, it is possible to use ceramics made from materials illustrated for the above-described porous layer 80. In the present embodiment, the first dense layer 92 and the second dense layer 96 are each assumed to be ceramics of alumina. The thickness of the first dense layer 92 may be e.g., 5 μm or greater and 40 μm or less. The thickness of the first dense layer 92 may be less than the thickness of the first inner porous layer 83. The thickness of the first dense layer 92 may be less than the thickness of the outer lead 75. The thickness of the first dense layer 92 may be less than or equal to 10 μm. The thickness of the first dense layer 92 is assumed to be the thickness measured at the portion located immediately above the outer lead 75 of the first dense layer 92. The porosity of each of the first dense layer 92 and the second dense layer 96 is preferably less than or equal to 8%, and more preferably less than or equal to 5%. For a lower porosity, the first dense layer 92 and the second dense layer 96 can further inhibit capillary action of water along the longitudinal direction of the element body 60.

In the longitudinal direction (here, the front-back direction) of the element body 60, the length Le1 (see FIG. 4) of the first dense layer 92 may be greater than or equal to 0.5 mm. The length Le1 may be greater than or equal to 5 mm. The length Le1 may be less than or equal to 25 mm, and may be less than or equal to 20 mm. In the present embodiment, the length of the high porosity region 77 of the outer lead 75 is equal to the length Le1 of the first dense layer 92.

The first dense layer 92 and the second dense layer 96 are each disposed to overlap with the inner peripheral surface of one of a plurality of insulators 44a to 44c in the position of the element body 60 along the longitudinal direction. In the present embodiment, as illustrated in FIG. 1, the first dense layer 92 and the second dense layer 96 are each disposed to overlap with the inner peripheral surface of the insulator 44b of the insulators 44a to 44c in the position of the sensor element 20 along the longitudinal direction. The inner peripheral surfaces of the insulator 44b are the surfaces thereof, opposed to the first dense layer 92 and the second dense layer 96, in other words, the surfaces exposed to the first dense layer 92 and the second dense layer 96, that is, the surfaces located on the upper side and the lower side of the inner peripheral surfaces of the cross-sectional rectangular shape of the insulator 44b.

Next, a method for producing the gas sensor 10 having the above-described structure will be described. A method for producing the sensor element 20 will be described, and then the method for producing the gas sensor 10 including the sensor element 20 installed therein will be described.

A method of manufacturing the sensor element 20 will be described. First, multiple (here, six) unfired ceramics green sheets corresponding to the element body 60 are prepared. Each green sheet is provided with notches, through-holes and grooves by a punching process as needed, and wiring patterns such as electrodes and the outer lead 75 are screen printed. Also, unfired porous layers which become the first inner porous layer 83 and the second inner porous layer 84 after firing, and unfired dense layers which become the first dense layer 92 and the second dense layer 96 after firing are formed by screen printing on the faces of the green sheets corresponding to the first, second faces 60a, 60b. Subsequently, multiple green sheets are stacked. The stacked multiple green sheets provide an unfired element body which becomes the element body after firing, and include an unfired porous layer and an unfired dense layer. Then the unfired element body is fired to obtain the element body 60 including the first inner porous layer 83, the second inner porous layer 84, the first dense layer 92, and the second dense layer 96. Subsequently, the outer porous layer 85 is formed by plasma spraying to obtain the sensor element 20. Note that as a method of manufacturing the porous layer 80, the first dense layer 92, and the second dense layer 96, the gel cast method, and dipping can be used in addition to screen printing and plasma spraying.

A wire pattern which becomes the outer lead 75 after firing can be formed by screen printing using the slurry obtained by kneading, for example, material particles (here, platinum particles and zirconia particles) of the outer lead 75, a pore forming material, and a solvent. The respective wire patterns of the low porosity region 76 and the high porosity region 77 of the outer lead 75 can be formed, for example, by preparing two types of slurries made from the same material except that both content ratios of the pore forming material are different, and screen printing the two types of slurries separately.

A method of manufacturing the gas sensor 10 with the built-in sensor element 20 will be described. First, the sensor element 20 is caused to penetrate a through-hole of the cylindrical body 41 in an axial direction, and the insulator 44a, the compact 45a, the insulator 44b, the compact 45b, an insulator 44c, and the metal ring 46 are disposed in this order between the inner peripheral surface of the cylindrical body 41 and the sensor element 20. Next, the metal ring 46 is pressed to compress the compacts 45a, 45b, and in the state, the element sealing member 40 is manufactured by forming the reduced diameter sections 43c, 43d, and the space between the inner peripheral surface of the cylindrical body 41 and the sensor element 20 is sealed. Subsequently, the protective cover 30 is welded to the element sealing member 40, and the nut 47 is attached thereto to obtain the assembly 15. The lead 55 that is passed through the rubber stopper 57, and the connector 50 connected to the lead 55 are prepared. The prepared connector 50 is connected to the back-end side of the sensor element 20, and the plurality of contact metal fittings 52 of the connector 50 are each electrically connected to a corresponding connector electrode of the plurality of upper connector electrodes 71 and lower connector electrodes 72. Subsequently, the external cylinder 48 is welded and fixed to the main metal fitting 42 to obtain the gas sensor 10.

Next, an example of the use of the gas sensor 10 having the above-described structure will be described. When the measurement-object gas flows through the pipe 58 with the gas sensor 10 attached to the pipe 58 as shown in FIG. 1, the measurement-object gas flows through the protective cover 30 and into the element chamber 33, and the front-end side of the sensor element 20 is exposed to the measurement-object gas. Then the measurement-object gas passes through the porous layer 80, reaches the outer electrode 64, and also reaches the sensor element 20 through the measurement-object gas inlet 61, and the detector 63 generates an electrical signal corresponding to the NOx concentration in the measurement-object gas as described above. By outputting this electrical signal through the upper and lower connector electrodes 71 and 72, the NOx concentration is detected based on the electrical signal.

In the sensor element 20 of thus configured gas sensor 10, as described above, the outer lead 75 has the low porosity region 76 covered by the first inner porous layer 83 of the porous layer 80, and the high porosity region 77 which is covered by the first dense layer 92, in contact with the low porosity region 76, and has a higher porosity than the low porosity region 76. Thus, the occurrence of crack of the sensor element 20 can be prevented. The inventors confirmed this by experiments and analysis. The reason why such an effect is obtained is probably as follows. First, the first dense layer 92 has a lower porosity and is denser than the first inner porous layer 83, thus has a high Young's modulus. Therefore, as compared to the first inner porous layer 83, the stress inside the first dense layer 92 is likely to increase. Thus, when the outer lead 75 is deformed, the stress difference is likely to occur between the first dense layer 92 and the first inner porous layer 83 (in particular, between the first dense layer 92 and the back end of the front-end side portion 83a, and between the first dense layer 92 and the front end of the back-end side portion 83b). When the stress difference increases too much, crack may occur in the sensor element 20, for example, crack occurs in the first dense layer 92 having a high Young's modulus. However, in the sensor element 20 of the present embodiment, the high porosity region 77 of the outer lead 75 covered by the first dense layer 92 has a higher porosity than the low porosity region 76, thus the high porosity region 77 has a lower Young's modulus than the low porosity region 76. Thus, in the presence of the high porosity region 77 having a low Young's modulus in a portion covered by the first dense layer 92 having a high Young's modulus, and in the presence of the low porosity region 76 having a high Young's modulus in a portion covered by the first inner porous layer 83 having a low Young's modulus, when the outer lead 75 is deformed, the difference in stress between the first dense layer 92 and the first inner porous layer 83 can be reduced. Thus, the occurrence of crack of the sensor element 20 can be probably prevented.

Note that deformation of the outer lead 75 is probably caused by e.g., thermal expansion of the outer lead 75 due to the heat from the heater 69 when the gas sensor 10 is used and/or a high temperature measurement-object gas. In the present embodiment, the outer lead 75 contains zirconia, thus TM transformation (change in the crystal structure from the T-phase (tetragonal crystal) to the M-phase (monoclinic crystal)) of zirconia occurs due to heat when the gas sensor 10 is used, and the volume of zirconia expands, which probably may cause deformation of the outer lead 75. In the sensor element 20 of the present embodiment, the occurrence of crack of the sensor element 20 due to deformation of the outer lead 75 like this can be prevented.

The measurement-object gas may contain water (water or sulfuric acid dissolved in water), and the water may move within the porous layer 80 due to capillary action. When the water reaches the upper, lower connector electrodes 71, 72, there is a possibility that rust or corrosion may occur in the upper, lower connector electrodes 71, 72, and/or the contact metal fittings 52 of the connector 50 in contact with the upper, lower connector electrodes 71, 72. However, in the gas sensor 10 of the present embodiment, even when water in the measurement-object gas moves toward the back-end side of the sensor element 20 (the element body 60) within the porous layer 80 (particularly, within the first inner porous layer 83 and within the second inner porous layer 84) due to capillary action, the water reaches the first, second dense layers 92, 96 before reaching the upper, lower connector electrodes 71, 72. Since the first, second dense layers 92, 96 are denser than the first, second inner porous layers 83, 84, capillary action of water along the longitudinal direction of the element body 60 is unlikely to occur. As a result, water is prevented from moving to the back-end side of the sensor element 20 with respect to the first, second dense layers 92, 96 and reaching the upper, lower connector electrodes 71, 72.

At this time, since the porosity of the high porosity region 77 covered by the first dense layer 92 is less than or equal to 40%, movement of water inside the high porosity region 77 can prevent the water from coming around the first dense layer 92 and moving to the back-end side of the sensor element 20 with respect to the first dense layer 92. Thus, water can be prevented from reaching the upper connector electrodes 71. Furthermore, when the porosity of the high porosity region 77 is less than 15%, water can be further prevented from moving through the inside of the high porosity region 77, thus it is possible to further prevent water from reaching the upper connector electrodes 71.

The first, second dense layers 92, 96 are each disposed to overlap with the inner peripheral surface of the insulator 44b in the position of the sensor element 20 (the element body 60) along the longitudinal direction, thus water can be prevented from coming around the first, second dense layers 92, 96 from the outside of the sensor element 20, and moving to the back-end side of the sensor element 20. For example, as a comparative embodiment, a case is considered where the first, second dense layers 92, 96 are each disposed to overlap with the inner peripheral surface of the compact 45a of FIG. 1 in the position of the sensor element 20 along the longitudinal direction. As mentioned above, the compact 45a is obtained by press-molding powder, and has water-absorbing properties. Thus, in the comparative embodiment, movement of water in the compact 45a may cause the water to come around the first, second dense layers 92, 96 from the outside of the sensor element 20, and move to the back-end side with respect to the first, second dense layers 92, 96. In contrast, in the sensor element 20 of the present embodiment, as illustrated in FIG. 1, the first, second dense layers 92, 96 are each disposed to overlap with the inner peripheral surface of the insulator 44b in the position of the sensor element 20 along the longitudinal direction, and the insulator 44b is dense. Thus, water can be prevented from coming around the multiple first, second dense layers 92, 96 from the outside of the sensor element 20, and moving to the back-end side of the sensor element 20. Thus, water can be prevented from reaching the upper, lower connector electrodes 71, 72.

The correspondence relationship between the components of the present embodiment and the components of the present invention will now be clarified. The sensor element 20 of the present embodiment corresponds to a sensor element of the present invention, the element body 60 corresponds to an element body, the first face 60a corresponds to a lateral face, the outer electrode 64 corresponds to an outer electrode, the upper connector electrodes 71 (71b) corresponds to connector electrodes, the outer lead 75 corresponds to an outer lead section, the porous layer 80 (particularly, the first inner porous layer 83) corresponds to a porous layer, the first dense layer 92 corresponds to a dense layer, the low porosity region 76 corresponds to a low porosity region, and the high porosity region 77 corresponds to a high porosity region.

In the sensor element 20 of the present embodiment described in detail above, the outer lead 75 has the low porosity region 76 covered by the first inner porous layer 83, and the high porosity region 77 which is covered by the first dense layer 92, in contact with the low porosity region 76, and has a higher porosity than the low porosity region 76. Thus, the occurrence of crack of the sensor element 20 can be prevented.

With the porosity of the high porosity region 77 less than or equal to 40%, movement of water inside the high porosity region 77 can prevent the water from coming around the first dense layer 92 and moving to the back-end side of the element body 60. Thus, water can be prevented from reaching the upper connector electrodes 71. Furthermore, with the porosity of the high porosity region 77 less than 15%, water can be further prevented from reaching the upper connector electrodes 71. From this point of view, the porosity of the high porosity region 77 is preferably less than or equal to 10%, and more preferably less than or equal to 7%.

In addition, with the difference in porosity greater than or equal to 1% between the high porosity region 77 and the low porosity region 76, the effect of preventing the occurrence of crack of the sensor element 20 is more reliably obtained by providing the high porosity region 77 and the low porosity region 76 mentioned above.

Furthermore, with the porosity of the high porosity region 77 greater than or equal to 5%, the effect of preventing the occurrence of crack of the sensor element 20 is more reliably obtained by providing the high porosity region 77 and the low porosity region 76 mentioned above. The reason for this is that when the porosity is greater than or equal to 5%, it is probable that the Young's modulus of the high porosity region 77 is sufficiently low.

With the thickness of the first dense layer 92 less than or equal to 10 μm, the occurrence of crack of the sensor element 20 can be further prevented. The reason for this is that with the thickness of the first dense layer 92 less than or equal to 10 μm which is relatively thin, the Young's modulus of the first dense layer 92 is reduced, and it is probable that the difference in stress between the first dense layer 92 and the first inner porous layer 83 can be further reduced.

Also, the outer lead 75 contains zirconia. When the outer lead 75 contains zirconia, the zirconia expands due to TM transformation, and the outer lead 75 is deformed, thus crack of the sensor element 20 tends to occur. Thus, it is significant to prevent the occurrence of crack of the sensor element 20 by providing the outer lead 75 with the low porosity region 76 and the high porosity region 77.

The present invention is not limited to the embodiment described above. It will be appreciated that the present invention can be implemented in various forms so long as they fall within the technical scope of the invention.

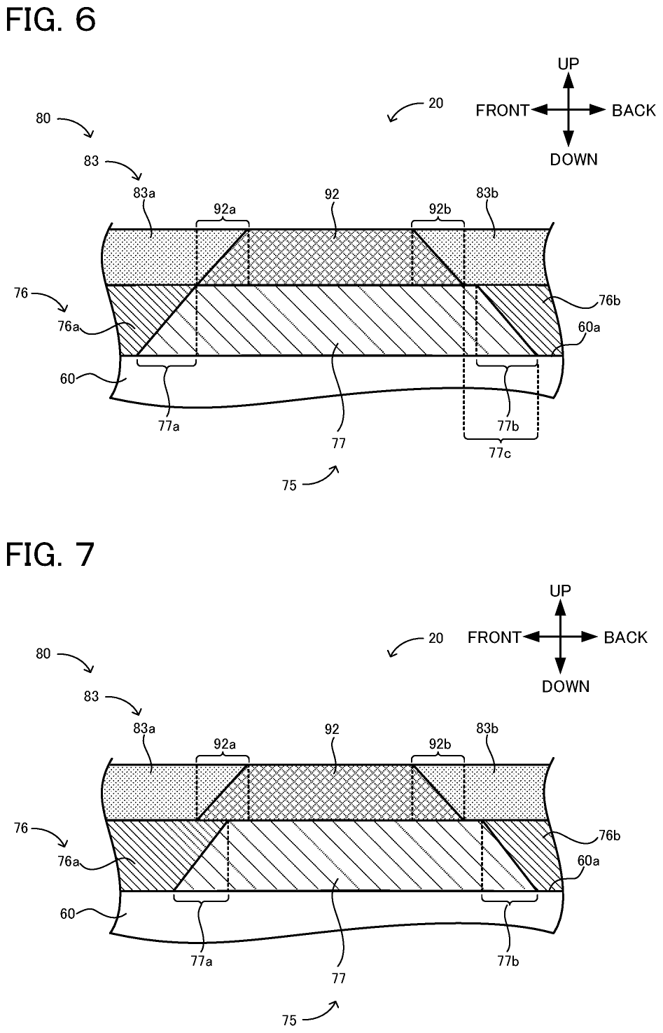

For example, in the above-described embodiment, in any contact portion between the first dense layer 92 and the first inner porous layer 83 illustrated in FIG. 5, there is no overlapping section between the first dense layer 92 and the first inner porous layer 83 in the thickness direction of the first dense layer 92, i.e., in the up-down direction. However, without being limited to this, the first dense layer 92 may have an overlapping section at a portion in contact with the first inner porous layer 83, the overlapping section overlapping with the first inner porous layer 83 in the thickness direction of the first dense layer 92. FIG. 6 is a cross-sectional view illustrating the periphery of a first dense layer 92 and a high porosity region 77 in a modification. In FIG. 6, the first dense layer 92 and the first inner porous layer 83 overlap in part with each other in the thickness direction of the first dense layer 92, i.e., in the up-down direction. Thus, the first dense layer 92 has an overlapping section 92a which is part of the front-end side of the first dense layer 92, and an overlapping section 92b which is part of the back-end side. The overlapping section 92a is a section of the first dense layer 92, the section overlapping with the back end of the front-end side portion 83a of the first inner porous layer 83 in the up-down direction, and the upper face of the overlapping section 92a is in contact with the front-end side portion 83a. The upper face of the overlapping section 92a is inclined with respect to the front-back direction, and the overlapping section 92a has a shape such that the closer to the front end thereof, the smaller the thickness. The overlapping section 92b is a section of the first dense layer 92, the section overlapping with the front end of the back-end side portion 83b of the first inner porous layer 83 in the up-down direction, and the upper face of the overlapping section 92b is in contact with the back-end side portion 83b. The upper face of the overlapping section 92b is inclined with respect to the front-back direction, and the overlapping section 92b has a shape such that the closer to the back end thereof, the smaller the thickness. In FIG. 6, the low porosity region 76 and the high porosity region 77 also overlap in part with each other in the thickness direction of the high porosity region 77, i.e., in the up-down direction. Thus, the high porosity region 77 has an overlapping section 77a which is part of the front-end side of the high porosity region 77, and an overlapping section 77b which is part of the back-end side. The overlapping section 77a is a section of the high porosity region 77, the section overlapping with the back end of the front-end side portion 76a of the low porosity region 76 in the up-down direction, and the upper face of the overlapping section 77a is in contact with the front-end side portion 76a. The upper face of the overlapping section 77a is inclined with respect to the front-back direction, and the overlapping section 77a has a shape such that the closer to the front end thereof, the smaller the thickness. The overlapping section 77b is a section of the high porosity region 77, the section overlapping with the front end of the back-end side portion 76b of the low porosity region 76 in the up-down direction, and the upper face of the overlapping section 77b is in contact with the back-end side portion 76b. The upper face of the overlapping section 77b is inclined with respect to the front-back direction, and the overlapping section 77b has a shape such that the closer to the back end thereof, the smaller the thickness. Such overlapping sections 77a, 77b, 92a, 92b may be formed due to, for example, manufacturing reasons of the sensor element 20. For example, in the above-described manufacturing method for the sensor element 20, when a pattern for the high porosity region 77 of the outer lead 75 is formed, then a pattern for the low porosity region 76 is formed by screen printing, the overlapping sections 77a, 77b are formed by portions of the front and the back of the high porosity region 77 being covered by the low porosity region 76 from the outer side (upper side) of the sensor element 20 as in FIG. 6. Similarly, when a pattern for the first dense layer 92 is formed, then a pattern for the first inner porous layer 83 is formed by screen printing, the overlapping sections 92a, 92b are formed as in FIG. 6.

Even when the overlapping sections 77a, 77b, 92a, 92b are provided as in FIG. 6, the outer lead 75 has the low porosity region 76 covered by the first inner porous layer 83, and the high porosity region 77 which is covered by the first dense layer 92, in contact with the low porosity region 76, and has a higher porosity than the low porosity region 76, thus the occurrence of crack of the sensor element 20 can be prevented as in the above-described embodiment. At this time, the first dense layer 92 preferably covers only the high porosity region 77 of the low porosity region 76 and the high porosity region 77 over a length from the front-end side to the back-end side of the first dense layer 92 including the overlapping sections 92a, 92b. In other words, it is preferable that the low porosity region 76 be not present immediately under the first dense layer 92 over a length from the front end to the back end of the first dense layer 92 in FIG. 6. In FIG. 6, the front-end position of the overlapping section 92a and the back-end position (=the back-end position of the front-end side portion 76a of the low porosity region 76) of the overlapping section 77a are at the same position in the front-back direction, thus the front-end side portion 76a of the low porosity region 76 is not present immediately under the overlapping section 92a. The front-end position (=the front-end position of the back-end side portion 76b of the low porosity region 76) of the overlapping section 77b is backward of the back-end position of the overlapping section 92b, thus the back-end side portion 76b of the low porosity region 76 is not present immediately under the overlapping section 92b. Because of these, the low porosity region 76 is not present immediately under the first dense layer 92 over a length from the front end to the back end of the first dense layer 92. Thus, the effect of preventing the occurrence of crack of the sensor element 20 is more reliably obtained by the low porosity region 76 and the high porosity region 77. For example, as illustrated in FIG. 7, part of the front-end side portion 76a of the low porosity region 76 may be present immediately under the overlapping section 92a of the first dense layer 92, in other words, the overlapping section 92a and the overlapping section 77a may be in an overlapping form in part in the up-down direction. As in FIG. 6 rather than the form in FIG. 7, it is preferable that the low porosity region 76 be not present immediately under the first dense layer 92 over a length from the front end to the back end of the first dense layer 92.

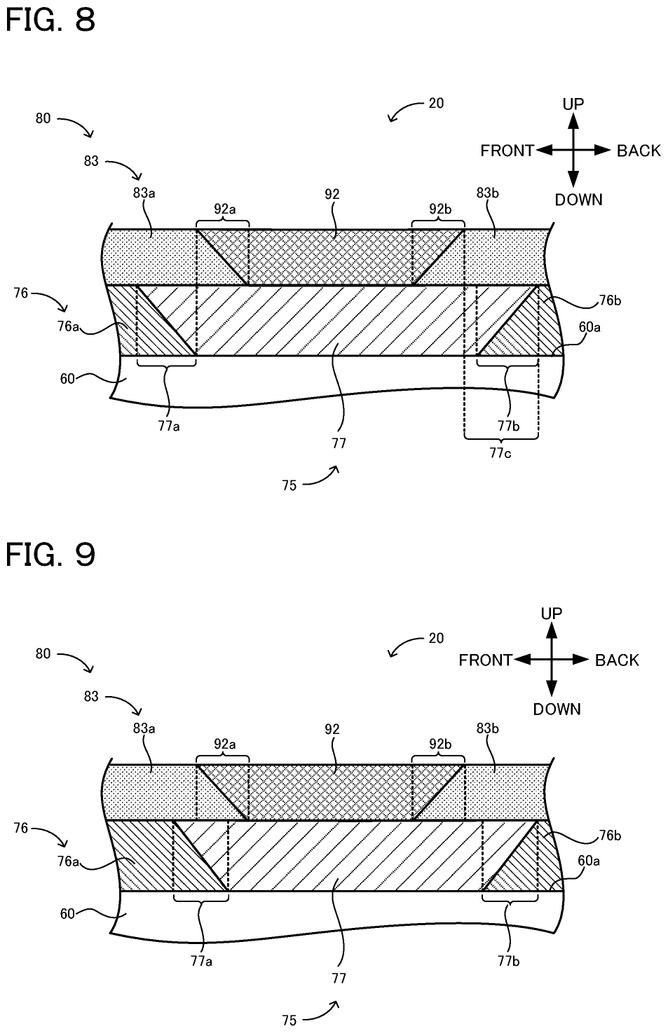

In FIG. 6, the first dense layer 92 and the first inner porous layer 83 overlap in part with each other so that the first inner porous layer 83 is located above; however, without being limited to this, both layers may overlap in part with each other so that the first dense layer 92 is located above the first inner porous layer 83. The same applies to the overlapping vertical relationship between the low porosity region 76 and the high porosity region 77. For example, FIG. 8 is a cross-sectional view when the overlapping vertical relationship between the first dense layer 92 and the first inner porous layer 83 is reversed, and the overlapping vertical relationship between the low porosity region 76 and the high porosity region 77 is reversed in FIG. 6. Similarly, FIG. 9 is a cross-sectional view when the overlapping vertical relationship in FIG. 7 is reversed. In FIG. 8 and FIG. 9, the same components as in FIG. 6 and FIG. 7 are labeled with the same symbol, and a detailed description is omitted. In FIG. 9, the lower face of the first dense layer 92 does not overlap with the overlapping section 77a of the high porosity region 77 in the up-down direction; however, the overlapping section 92a of the first dense layer 92 overlaps in part with the overlapping section 77a of the high porosity region 77 in the up-down direction. In other words, in FIG. 9, as in FIG. 7, part of the front-end side portion 76a of the low porosity region 76 is present immediately under the overlapping section 92a of the first dense layer 92. In contrast, in FIG. 8, the overlapping section 92a of the first dense layer 92 does not overlap with the overlapping section 77a of the high porosity region 77 in the up-down direction. In other words, in FIG. 8, as in FIG. 6, the front-end side portion 76a of the low porosity region 76 is not present immediately under the overlapping section 92a. Thus, in FIG. 8, the low porosity region 76 is not present immediately under the first dense layer 92 over a length from the front end to the back end of the first dense layer 92. Therefore, the configuration of FIG. 8 is more preferable than the configuration of FIG. 9.

In the above-described embodiment, the high porosity region 77 illustrated in FIG. 5 does not have a protruding portion which protrudes from the first dense layer 92 to the front-end side and/or the back-end side; however, the high porosity region 77 may have a protruding portion. For example, in the high porosity region 77 of FIG. 6 and FIG. 8, the overlapping section 77a protrudes from the first dense layer 92 to the front-end side, and the overlapping section 77a corresponds to a protruding portion. The high porosity region 77 of FIG. 6 and FIG. 8 has a region 77c which protrudes from the first dense layer 92 to the back-end side. The region 77c includes the overlapping section 77b, and the region between the overlapping section 92b and the overlapping section 77b in the front-back direction. The region 77c also corresponds to a protruding portion. Like this, even when the high porosity region 77 has protruding portions (the overlapping section 77a, the region 77c), the outer lead 75 has the low porosity region 76 covered by the first inner porous layer 83, and the high porosity region 77 which is covered by the first dense layer 92, in contact with the low porosity region 76, and has a higher porosity than the low porosity region 76, thus the occurrence of crack of the sensor element 20 can be prevented as in the above-described embodiment. At this time, it is preferable that the length of each protruding portion in the front-back direction be less than or equal to 0.5 mm. In the case of FIG. 6 and FIG. 8, it is preferable that the length of the overlapping section 77a in the front-back direction and the length of the region 77c in the front-back direction each be less than or equal to 0.5 mm. In this configuration, the effect of preventing the occurrence of crack of the sensor element 20 is more reliably obtained. From the viewpoint of the effect, it is more preferable that the length of each protruding portion in the front-back direction be less than or equal to 0.3 mm.

In the above-described embodiment, the first inner porous layer 83 of the sensor element 20 covers the first face 60a excluding the region where the upper connector electrodes 71 and the first dense layer 92 are present, but is not limited thereto. For example, the first inner porous layer 83 may cover the region from the front end of the first face 60a to the front end of the upper connector electrodes 71 of the first face 60a or to a predetermined position front of the front end. Also, of the first inner porous layer 83, a gap region (region where neither a porous layer nor a dense layer is present) may be provided between the front-end side portion 83a and the first dense layer 92 or between the back-end side portion 83b and the first dense layer 92. Alternatively, the first inner porous layer 83 may not include the back-end side portion 83b, and the region between the first dense layer 92 and the upper connector electrodes 71 may serve as a gap region. The outer lead 75 may be exposed to space where a gap region is provided. The low porosity region of the outer lead may be disposed to be covered by the porous layer in contact with the dense layer, and the high porosity region may be disposed to be in contact with the low porosity region and covered by the dense layer. Thus, for example, when a gap region is provided between the back-end side portion 83b of the first inner porous layer 83 and the first dense layer 92, or when the back-end side portion 83b is not present, the porous layer in contact with the first dense layer 92 is only the front-end side portion 83a. In this case, of the outer lead 75, the front-end side portion 76a covered by the front-end side portion 83a only needs to be a low porosity region, and the back-end side portion 76b of the outer lead 75 does not need to be a low porosity region. For example, the back-end side portion 76b may have the same porosity as that of the high porosity region 77.

In the above-described embodiment, the first dense layer 92 overlaps with the insulator 44b in the position in the front-back direction, but is not limited thereto. For example, the first dense layer 92 may overlap with the insulator 44a or the insulator 44c in the position in the front-back direction.

In the above-described embodiment, the sensor element 20 includes the outer porous layer 85, but may not include it.

In the above-described embodiment, the element body 60 is in a rectangular parallelepiped shape, but is not limited thereto. For example, the element body 60 may have a tubular shape or a cylindrical shape. In this case, the element body 60 has only one lateral face.

In the above-described embodiment, the gas sensor 10 includes two compacts 45a, 45b, and three insulators 44a to 44c, but may include one or a plurality of compacts, and a plurality of insulators (dense bodies). For example, a configuration with the insulator 44b removed from the gas sensor 10 may be adopted. In this case, the compacts 45a, 45b are configured to be adjacent to each other in the front-back direction, thus the compacts 45a, 45b may be integrated.

### EXAMPLES

Hereinafter, an example in which a sensor element is specifically produced will be described as an example. Experimental Examples 2 to 4, 6, 7, 9, 10, 12, 13 correspond to examples of the present invention, and Experimental Examples 1, 5, 8, 11 correspond to Comparative Examples. Note that the present invention is not limited to the following examples.

### Experimental Examples 1 to 13

Respective sensor elements were produced by the same manufacturing method for the gas sensor 10 in FIG. 1 to FIG. 6 to provide Experimental Examples 1 to 13. The dimensions of the element bodies 60 of these sensor elements 20 are such that the length is 52.5 mm, the width is 4.25 mm, and the thickness is 1.45 mm. The first dense layer 92 is assumed to have length Le1 of 5 mm, a thickness of 10 μm, and a porosity of 0%. The outer lead 75 is assumed to have a line width of 0.4 mm, and a line thickness of 15 μm. Hereinafter, of the outer lead 75, the region at a position corresponding to the high porosity region 77 may be referred to as the first region, and the region at a position corresponding to the low porosity region 76 may be referred to as the second region. In Experimental Examples 1 to 13, the porosities of the first region and the second region of the outer lead 75 were changed in various ways as illustrated in Table 1. In Experimental Examples 2 to 4, 6, 7, 9, 10, 12, 13, the porosity of the first region of the outer lead 75 was set higher than the porosity of the second region so that the outer lead 75 has the low porosity region 76 and the high porosity region 77. In Experimental Examples 1, 5, 8, 11, the porosity of the first region of the outer lead 75 was set less than or equal to the porosity of the second region.

TABLE 1

| | Porosity of the | | | |
|---|---|---|---|---|
| | outer lead[%] | | | |
| | First | Second | | |
| | region | region | | Evaluation |
| | (high | (low | Evaluation | of Liquid |
| | porosity | porosity | of Crack | Immersion |
| | region) | region) | Test | Test |
| Experimental Examples 1 | 5 | 5 | F | A |
| Experimental Examples 2 | 5 | 4 | A | A |
| Experimental Examples 3 | 6 | 5 | A | A |
| Experimental Examples 4 | 7 | 5 | A | A |
| Experimental Examples 5 | 5 | 6 | F | A |
| Experimental Examples 6 | 15 | 14 | A | B |
| Experimental Examples 7 | 21 | 20 | A | B |
| Experimental Examples 8 | 20 | 21 | F | B |
| Experimental Examples 9 | 31 | 30 | A | B |
| Experimental Examples 10 | 40 | 39 | A | B |
| Experimental Examples 11 | 40 | 40 | F | B |
| Experimental Examples 12 | 41 | 40 | A | F |
| Experimental Examples 13 | 42 | 40 | A | F |

### [Crack Test]

For each sensor element 20 in Experimental Examples 1 to 13, a test to evaluate crack resistance was conducted. Specifically, first, 20 units of the sensor element 20 of Experimental Example 1 were prepared, and placed in an autoclave in saturated water vapor at a temperature of 180° C. and were left for five hours. Subsequently, for each of the 20 units of the sensor element 20, it was visually checked using an optical microscope whether crack has occurred. The ratio of the number of sensor elements 20 in which crack has occurred to the 20 units of the sensor element 20 was calculated as a crack rate. For each sensor element 20 in Experimental Examples 2 to 13, the crack rate was calculated similarly. The crack rate in Experimental Example 1 was set to a reference value, and when the crack rate was less than the reference value in Experimental Examples 1 to 13, determination of excellent (A) was made, when the crack rate was higher than or equal to the reference value, determination of fail (F) was made.

### [Liquid Immersion Test]

For each sensor element 20 in Experimental Examples 1 to 13, a liquid immersion test was conducted in which upon immersion of the front-end side of the element body 60 into liquid, the degree of imbibition of the liquid into the back-end side of the element body 60 due to capillary action was examined. First, with the front-end side of the sensor element 20 facing down and the longitudinal direction of the sensor element 20 in the vertical direction, a portion of the sensor element 20 was immersed in the red check liquid, the portion being from a position backward of the front end the outer lead 75 of the sensor element 20 to a predetermined position (hereinafter, an immersion position) front of the first dense layer 92. The sensor element 20 was left unattended for 24 hours in this state, and an arrival position indicating how far the red check liquid has reached backward of the immersion position was visually measured. The arrival position was measured as a distance from the front end of the element body 60. When the arrival position after 24 hours was front of the back end of the first dense layer 92, in other words, when the red check liquid has not passed through the first dense layer 92, determination of excellent (A) was made. When the arrival position was backward of the first dense layer 92, but has not passed a predetermined position which is front of the front end of the upper connector electrodes 71, determination of good (B) was made. When the arrival position has passed through the predetermined position, determination of fail (F) was made. As the red check liquid, Stamp Ink (exclusive use for sol ink pad) (product model number: S-1, color type: red) manufactured by Shachihata Inc. was used. The red check liquid contains water of 50 to 60 wt %, glycerin of 30 to 40 wt %, and dye of 5 to 15 wt %. The components and composition of the red check liquid are stated in Safe Data Sheet (SDS) of Shachihata Inc.

As seen from Table 1, in each of Experimental Examples 2 to 4, 6, 7, 9, 10, 12, 13 in which the porosity of the first region of the outer lead 75 is higher than the porosity of the second region, in other words, the low porosity region 76 and the high porosity region 77 are provided in the crack test, the evaluation was excellent (A). In contrast, in each of Experimental Examples 1, 5, 8, 11 in which the porosity of the first region of the outer lead 75 is less than or equal to the porosity of the second region, the evaluation was fail (F). Based on the foregoing, it has been confirmed that the occurrence of crack of the sensor element 20 can be prevented with the outer lead 75 having the low porosity region 76 covered by the first inner porous layer 83, and the high porosity region 77 which is covered by the first dense layer 92, in contact with the low porosity region 76, and has a higher porosity than the low porosity region 76.

As seen from Table 1, from the mutual comparison between Experimental Examples 2 to 4, 6, 7, 9, 10, 12, 13 in which the evaluation of the crack test in the liquid immersion test was excellent (A), in Experimental Examples 12, 13 in which the porosity of the high porosity region 77 exceeds 40%, the evaluation was fail (F), whereas in Experimental Examples 2 to 4, 6, 7, 9, 10 in which the porosity of the high porosity region 77 is less than or equal 40%, the evaluation was good (B) or excellent (A). Based on the foregoing, when the porosity of the high porosity region 77 is less than or equal 40%, as compared to when the porosity exceeds 40%, it has been confirmed that water can be prevented from reaching the upper connector electrodes 71. Furthermore, between Experimental Examples 2 to 4, 6, 7, 9, 10, in Experimental Examples 6, 7, 9, 10 in which the porosity of the high porosity region 77 is greater than or equal 15%, the evaluation was good (B), whereas in Experimental Examples 2 to 4 in which the porosity of the high porosity region 77 is less than 15%, the evaluation was excellent (A). Based on the foregoing, when the porosity of the high porosity region 77 is less than 15%, as compared to when the porosity is 15% or greater and 40% or less, it has been confirmed that water can be prevented from reaching the upper connector electrodes 71.

The present application claims priority from Japanese Patent Application No. 2023-058160 filed Mar. 31, 2023, the entire contents of which are incorporated herein by reference.

What is claimed is:

1. A sensor element for detecting a specific gas concentration in a measurement-object gas, the sensor element comprising:
an element body having a front end and a back end which are both ends along a longitudinal direction, and a lateral face which is a surface along the longitudinal direction, wherein a front-end side is exposed to the measurement-object gas;
an outer electrode disposed on the front-end side of the lateral face;
a connector electrode disposed on a back-end side of the lateral face to be electrically connected to an outside;
an outer lead section disposed on the lateral face to electrically connect the outer electrode and the connector electrode;
a porous layer that covers part of the lateral face; and
a dense layer that covers part of the lateral face, the dense layer being disposed on the back-end side with respect to at least part of the porous layer, disposed on the front-end side with respect to the connector electrode, and in contact with the porous layer on the front-end side and/or the back-end side,
wherein the outer lead section has a low porosity region covered by the porous layer, and a high porosity region which is covered by the dense layer, in contact with the low porosity region, and has a higher porosity than the low porosity region.

2. The sensor element according to claim 1,
wherein the high porosity region has a porosity less than or equal to 40%.

3. The sensor element according to claim 1,
wherein the high porosity region has a porosity less than 15%.

4. The sensor element according to claim 1,
wherein a difference in porosity between the high porosity region and the low porosity region is greater than or equal to 1%.

5. The sensor element according to claim 1,
wherein the high porosity region has a porosity greater than or equal to 5%.

6. The sensor element according to claim 1,
wherein the dense layer has a thickness less than or equal to 10 μm.

7. The sensor element according to claim 1,
wherein the dense layer has an overlapping section at a portion in contact with the porous layer, the overlapping section overlapping with the porous layer in a thickness direction of the dense layer, and
the dense layer covers only the high porosity region of the high porosity region and the low porosity region over a length from the front-end side to the back-end side of the dense layer including the overlapping section.

8. The sensor element according to claim 1,
wherein the high porosity region has a protruding portion which protrudes from the dense layer to the front-end side and/or the back-end side, the protruding portion being covered by the porous layer, and
the protruding portion has a length less than or equal to 0.5 mm along the longitudinal direction.

9. The sensor element according to claim 1,
wherein the outer lead section contains zirconia.

10. A gas sensor comprising the sensor element according to claim 1.

11. The sensor element according to claim 2,
wherein a difference in porosity between the high porosity region and the low porosity region is greater than or equal to 1%.

12. The sensor element according to claim 3,
wherein a difference in porosity between the high porosity region and the low porosity region is greater than or equal to 1%.

13. The sensor element according to claim 2,
wherein the high porosity region has a porosity greater than or equal to 5%.

14. The sensor element according to claim 3,
wherein the high porosity region has a porosity greater than or equal to 5%.

15. The sensor element according to claim 4,
wherein the high porosity region has a porosity greater than or equal to 5%.

16. The sensor element according to claim 11,
wherein the high porosity region has a porosity greater than or equal to 5%.

17. The sensor element according to claim 12,
wherein the high porosity region has a porosity greater than or equal to 5.

18. The sensor element according to claim 17,
wherein the dense layer has a thickness less than or equal to 10 μm.
